(19) United States
(12) Patent Application Publication
(10) Pub. No.: US 2012/0201759 A1
(43) Pub. Date: Aug. 9, 2012

(54) TUNABLE MULTISCALE STRUCTURES COMPRISING BRISTLY, HOLLOW METAL/METAL OXIDE PARTICLES, METHODS OF MAKING AND ARTICLES INCORPORATING THE STRUCTURES

(75) Inventors: Christopher Jameson Love, Atlantis, FL (US); J. David Smith, Cambridge, MA (US); Kripa K. Varanasi, Lexington, MA (US)

(73) Assignee: Massachusetts Institute of Technology, Cambridge, MA (US)

(21) Appl. No.: 13/363,706

(22) Filed: Feb. 1, 2012

Related U.S. Application Data
(60) Provisional application No. 61/439,030, filed on Feb. 3, 2011.
(60) Provisional application No. 61/493,549, filed on Jun. 6, 2011.
(60) Provisional application No. 61/547,852, filed on Oct. 17, 2011.

(51) Int. Cl. B32B 3/00; B05D 7/00; B05D 3/02; B22F 1/02; B32B 5/16; F28D 19/00; C01B 13/32; C09K 5/00; B01J 35/02; C09D 1/00; A61B 5/055; G01N 33/00; F28F 1/00; H01M 6/36; H01M 4/02; H01M 4/04; H01G 9/155; B82Y 40/00; B82Y 30/00

(52) U.S. Cl. 424/9.32; 361/502; 427/212; 419/19; 428/403; 428/141; 165/10; 423/592.1; 252/71; 502/300; 106/286.7; 73/23.2; 165/177; 429/112; 429/209; 977/890; 977/762

(57) ABSTRACT

Hierarchical nanostructures and methods of fabrication. The structures include particles having a metal oxide outer shell with metal oxide wires extending from the outer shell. A multiscale structure according to the invention has particles above and below a critical size wherein the particles above the critical size have wires extending from the surface. These structures may be fabricated from a mixture prepared of relatively smaller metal particles having a size threshold below a threshold for nanowire formation and of relatively larger metal particles having a size above the threshold for nanowire formation. The mixture is oxidized at a selected temperature and for a selected time whereby the relatively smaller particles sinter and nanowires grow on the relatively larger particles thereby creating tunable hierarchical structures with metal-to-metal contact between the particles.

This application claims priority to U.S. provisional patent application Ser. No. 61/439,030 filed Feb. 3, 2011; Ser. No. 61/493,549 filed Jun. 6, 2011; and Ser. No. 61/547,852 filed Oct. 18, 2011. The contents of these three provisional applications are incorporated herein by reference.

This invention was made with government support under Grant No. N66001-10-1-4047 awarded by the Space and Naval Warfare Systems Center and under Grant No. DGE0645960 awarded by the National Science Foundation. The government has certain rights in this invention.

### BACKGROUND OF THE INVENTION

This invention relates to the thermal oxidation of metals such as copper and iron, and more particularly to formation of nanowires on metal particles that can form hierarchical structures.

Metal oxide nanowires are useful in a variety of applications including gas-sensors¹, nanoelectronics², energy harvesting³, and photonics⁴. As semiconductors, their unique one-dimensionality; strong photon, phonon, and electron confinement; and predictable electrical and optical functionalities have made them widely applicable⁵. Many methods are available to form the nanowires including template-directed, vapor phase, solution phase, vapor-liquid-solid (VLS), and epitaxial growth⁶⁻¹⁰. We are interested in the thermal oxidation of metals, which is one of the simpler and more scalable approaches.

Copper oxide has been studied for its electronic properties¹¹ and has found additional applications as a photocathode, superhydrophobic surface^{12, 13}, and oxygen carrier in chemical looping combustion¹⁴. Among various metal oxides, copper oxide has shown the best oxidation/reduction performance for solid-coal chemical looping combustion¹⁴. The good catalytic properties of copper oxide have also been demonstrated in the oxidation of methane¹⁵ and a plasma-enhanced carbon monoxide oxidation process¹⁶.

Copper oxide nanowires can be formed in a straightforward manner by thermal oxidation of copper substrates including foils, grids, and wires¹⁷⁻²⁰. The formation of nanowires has been shown to occur at temperatures of 400-700° C.^{18, 19}. Copper particles are typically sintered under an inert atmosphere for thermal management applications such as heat pipes²¹⁻²⁴. Previous research²⁵ demonstrated that the selection of the appropriate particle size, porosity, and thickness of the sintered metal structure could promote thin film evaporation and maximization of heat transfer. Porous layer coatings of copper²⁶ and copper nanowire structures²⁷ have also been shown to enhance pool boiling critical heat flux over plain surfaces.

An object of the invention is the thermal oxidation of copper and other metal particles resulting in nanowire growth, a hollow interior, and the ability to create tunable and scalable hierarchical structures. By changing the particle size, we demonstrate for the first time the ability to control the extent of nanowire growth and identify regimes where many nanowires and almost no nanowires form. Systematic thermogravimetric analysis (TGA) and in-situ x-ray diffraction (XRD) experiments of thermal oxidation were conducted on particles of various sizes to understand the size-dependent process. We propose a mechanism for the reaction and suggest applications based on this new process of tunable nanowire growth.

### SUMMARY OF THE INVENTION

In a first aspect, the invention is a particle having a metal oxide outer shell with metal oxide wires extending from the outer shell. In another aspect, the invention is a multiscale structure including particles above and below a critical size wherein the particles above the critical size have wires extending from the surface. In a preferred embodiment the particles both above and below the critical size are in intimate metal-to-metal contact.

In another aspect, the invention disclosed herein is a method for making hierarchical structures including preparing a mixture of relatively smaller metal particles having a size below a threshold for nanowire formation and of relatively larger metal particles having a size above a threshold for nanowire formation. The mixture is oxidized at a selected temperature and for a selected time whereby the relatively smaller particles sinter and nanowires grow on the relatively larger particles thereby creating tunable hierarchical structures. In a preferred embodiment of this aspect of the invention the particles are copper or iron. Other metals with multiple oxidization states and a lattice mismatch with its oxide are suitable for nanowire growth under thermal oxidation conditions. In a preferred embodiment using copper, the threshold is in a range of approximately 2-4 μm with a preferred value around 3 μm. Suitable oxidation temperatures are in the range of 400-700° C. A preferred temperature is approximately 600° C. Suitable thermal oxidation times are in the range of approximately 30 minutes to 60 minutes.

In another aspect, the invention is a method for controlling the extent of nanowire growth on a metal particle including selecting a starting particle size with respect to a size threshold for nanowire growth and oxidizing the particle at a selected temperature and for a selected time whereby a desired extent of nanowire growth is achieved. In yet another aspect, the invention is a method for making a hollow structure comprising thermally oxidizing a metal particle. The hierarchical nanostructures made according the method disclosed herein will be described below.

### BRIEF DESCRIPTION OF THE DRAWING

FIGS. 1 a, b and c are SEM micrographs of copper particles in ambient air.

FIG. 2 is a graph of weight versus time showing a thermogravimetric (TGA) analysis of differently sized copper particles and bulk foil samples oxidized in air at 600° C.

FIGS. 3a, b, c and d are graphs of mass percentage versus time of in situ XRD time scans of copper samples.

FIGS. 4a and 4b are SEM images of a focused ion beam-milled cross section of a 1 μm (a) and 10 μm (b) oxidized copper particle.

FIGS. 5a and 5b are SEM micrographs showing tunable hierarchical structures produced using size-dependant nanowire growth.

FIGS. 6a, b, c and d are SEM micrographs showing the thermal oxidation of iron particles having a size in the range of 1-3 μm.

FIGS. 7a, b, c are schematic illustrations showing an oxidized network of partially sintered metal particles.

### DESCRIPTION OF THE PREFERRED EMBODIMENT

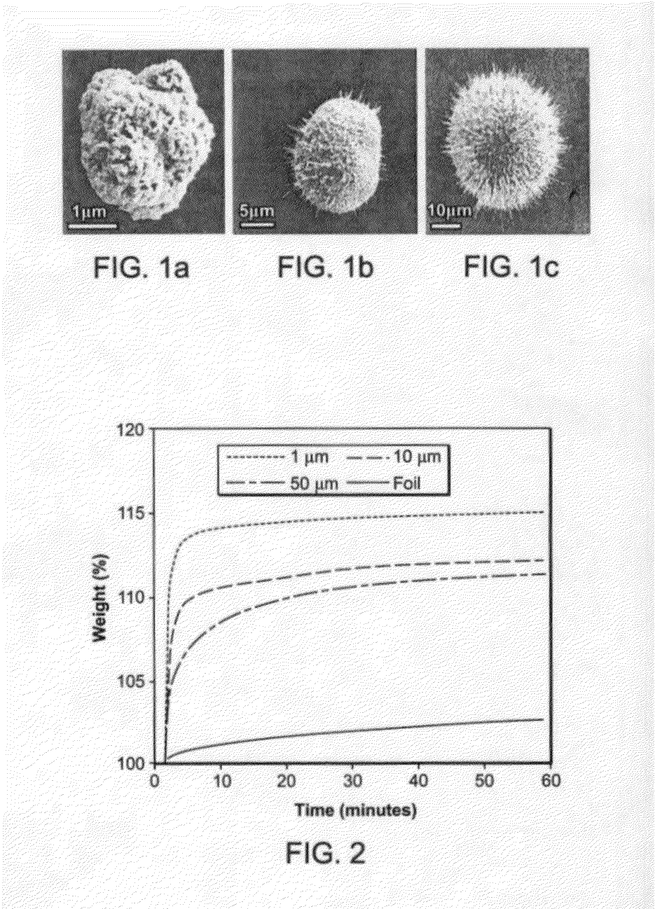

With reference to FIG. 1, prior to thermal oxidation, copper particles of 1, 10, and 50 μm average size were absent of nanowires. After thermal treatment in a furnace, all samples were black in color. Characterization on a field emission scanning electron microscope (FESEM, Zeiss Ultra55) revealed nanowire protrusions from spherical particles. Each nanowire grew approximately perpendicular to the surface, most of the wires were straight, and there was a significant variation in nanowire length. We did not observe branching or entanglement of the nanowires. Based on previous reports^{19, 28}, we expect the nanowires to be monoclinic CuO. The particles were roughened by the thermal oxidation process yielding nanoscale indentations. A comparison of the nanowire coverage on particles of different size for an oxidation temperature of 600° C. is shown in FIG. 1 and reveals a difference in nanowire growth based on the particle size. We observe a critical size of around 3 μm above which nanowires form, but the transition is stochastic; therefore, we specify two distinct regimes of nanowire growth: particle sizes of around 3 μm and below with no noticeable nanowire coverage and particle sizes of 10 μm and larger with appreciable nanowire coverage. Furthermore, particles with an average size ˜1 μm tend to agglomerate and form dense, sintered structures even without prior pressing or packing. At 400° C. and 500° C., we have seen a similar clear transition in nanowire growth on particle sizes between 3-10 μm. The influence of temperature on nanowire growth has been noted previously¹⁸. Within the reported^{18, 19} temperature range of 400-700° C. of nanowire growth, the effect of temperature was much less pronounced than that of the particle size. Furthermore, we did not observe a consistent trend in nanowire growth with temperature.

The growth mechanism of nanowires by thermal oxidation is not well understood in the literature. Most literature studies have proposed a mechanism of copper oxidation and nanowire growth based on two-steps^{18, 19}:

4Cu+O₂→2Cu₂O

2Cu₂O+O₂→4CuO

The growth of nanowires involves the formation of a major product (Cu₂O) that acts as a precursor to the rate-determining second reaction. X-ray diffraction (XRD) patterns of oxidized copper samples have supported this mechanism by revealing a strong signal for Cu₂O with a much smaller peak corresponding to CuO^{18, 19}. The Cu₂O layer has been proposed²⁹ to be a seed for CuO nanowire growth. After removal of the Cu₂O phase, no increase in nanowire length and diameter was observed²⁹ with increasing duration at the same temperature. Furthermore, it has been suggested³⁰ that the morphology of the outer nanowire “layer” is determined by the microstructure of the underlying Cu₂O layer.

The most recent literature and proposed models suggests two key ideas for nanowire growth to occur: outward copper ion diffusion through a defective Cu₂O layer³⁰ and a critical compressive stress between Cu₂O/CuO²⁸. Other models^{19, 31} involving the evaporation and recondensation of oxide species (vapor-solid model) have not been well-supported^{20, 28, 30}. To understand the observed size-dependent behavior and determine if it follows the proposed mechanisms, we monitored bulk oxidation rate by thermogravimetric analysis (TGA Q50, TA Instruments) at 600° C. for 1 hr in air. From TGA (FIG. 2), we observe that smaller particles undergo oxidation at a much faster rate than larger particles with a consistent trend of increasing rate with decreasing size. As expected, the foil sample has the slowest kinetics. The higher rate of oxidation of the smaller particles is presumably due to their larger specific surface area.

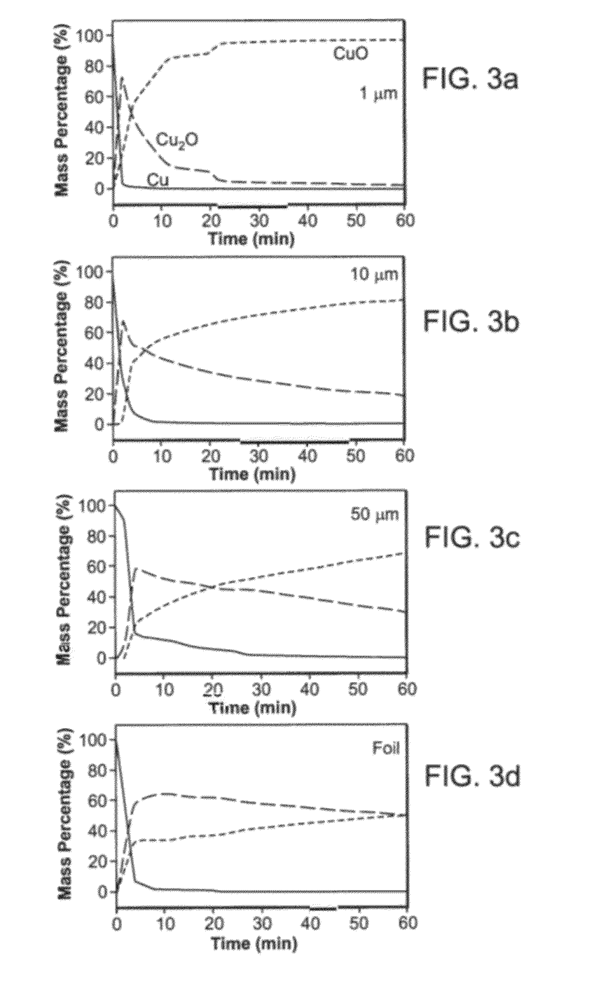

In order to further understand the size-dependent results in our SEM and TGA experiments, we examined the evolution of the copper and copper oxide phases with time by conducting a detailed in-situ XRD (PANalytical X'Pert Pro) experiment. The oxidation was conducted at 600° C. in an atmosphere consisting of 5% oxygen and 95% nitrogen in order to slow down the rate of the reaction and capture the evolution of the initial process. A Rietveld analysis was performed on the raw data to determine changes in mass percent of each compound, which are shown in FIG. 3. For all particle sizes in FIG. 3, we observe the expected peaks for Cu, Cu₂O, and CuO, though considerable difference is seen in the evolution of these compounds based on the size as discussed below.

Our results support the general mechanism of nanowire formation based on the availability of copper ions and an optimal oxide shell. By examining three parameters copper availability, Cu₂O relative amount, and CuO thickness, we hypothesize that oxidation of smaller particles does not result in the formation of an oxide shell that reaches a critical stress level needed for nanowire growth²⁸. Furthermore, the faster rate of oxidation of smaller particles rapidly depletes the Cu₂O shell, thereby eliminating the short-circuit³² (lower activation energy) diffusion paths needed for nanowire growth.

In-situ XRD reveals a dependence of the relative amounts of Cu, Cu₂O, and CuO on initial particle size. As observed in the TGA experiment, the rate of formation of copper oxide increases with decreasing particle size. Within only the first minute, copper is completely depleted on the 1 μm size powder. Likewise, the Cu₂O phase disappears after 20-30 minutes on the 1 μm powder but around 30-60% remains on the larger sizes after the same amount of time. Based on these XRD results, we explain the size-dependent nanowire growth by looking at the three essential conditions required for nanowire growth: Cu availability, Cu₂O formation, and CuO thickness. A more rapidly depleted Cu₂O layer on smaller sized particles combined with copper depletion in the particle core eliminates the possibility for substantial copper diffusion through a defective Cu₂O layer that was proposed as essential to nanowire formation. Furthermore, we expect that a sufficiently thick CuO layer did not form on the 1 μm particles to achieve a critical level of stress that has been suggested²⁸ for the promotion of nanowires. An approximate calculation of the initial particle size necessary to achieve a critical oxide thickness for nanowire growth is ˜2-4 μm, which is consistent with our observed results of little to no nanowire growth on 1 μm sized particles oxidized at 600° C. It is expected that the reaction rates are even faster at ambient 21% oxygen, further supporting our observed size-dependent results in FIG. 1.

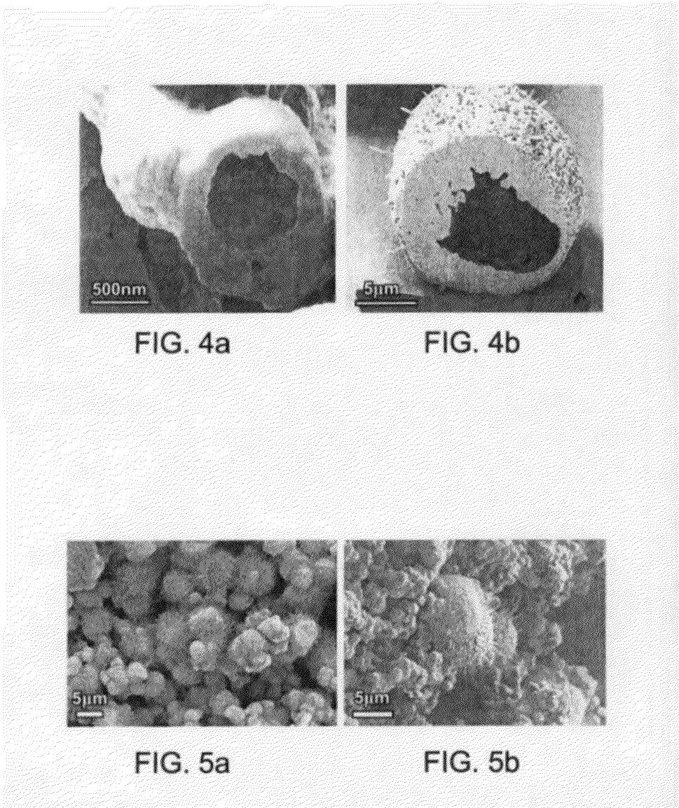

Given our hypothesis of outward copper diffusion from a spherical core and the observation³³ that such a process led to inner pores and void formation in cobalt or nickel-plated beryllium powder, we checked for void formation with a dual-beam focused ion beam (FIB) and SEM (Zeiss NVision 40). After cutting open and imaging the particles, we discovered that the oxidized particles are hollow (FIG. 4). In comparison, the unoxidized particles are solid. The observed void formation could be explained by the Kirkendall effect, which has been reported to cause voids in the oxidation of cobalt nanoparticles³⁴. The preformed surface oxide provides defects for metal-out diffusion from the particle core to the surface region where oxygen anions are relatively immobile^{18, 35}. If the inwardly migrating vacancies become supersaturated in the core, then they coalesce into a single void. For studies³³ on 30 μm cobalt or nickel-plated beryllium powder, the Kirkendall effect led to a large volume fraction of pores but without the intact outer shell observed in our samples. Our FIB results confirm the proposed mechanism of outwardly diffusing copper atoms, which leads to the formation of a central void and hollow particles.

The structures, composed of metal particles surrounded by metal oxide nanowires, can be applied to catalysis, chemical looping combustion, and thermal management applications including heat pipes, boilers, and electronics cooling. The size-dependent oxidation could be extended to optimize oxygen carriers in chemical looping combustion. Furthermore, the high surface area of nanowire-covered particles with inner voids might be used to enhance catalytic activity. Size-dependent kinetics is also useful in sintering and the creation of wicking structures where a controllable porosity is desired.

We disclose herein multiscale or hierarchical structures and a straightforward process to synthesize these new hierarchical structures through a tunable growth process. By mixing small and large particles, wherein smaller particles readily sinter and nanowires grow only on larger particles, we created the hierarchical structures as shown in FIG. 5. Such hierarchical structures can be used to improve the wicking ability and fluid transport in heat pipes and enhance boiling heat transfer. With these structures, we have demonstrated enhanced heat transfer in spray-cooling applications³⁶. Here we have identified a simple approach to fabricate hollow inorganic particles, which could be used for encapsulation³⁷ and to make new kinds of hybrid materials for catalytic or biological applications. Future study will investigate size-dependent properties in other metal-oxide systems.

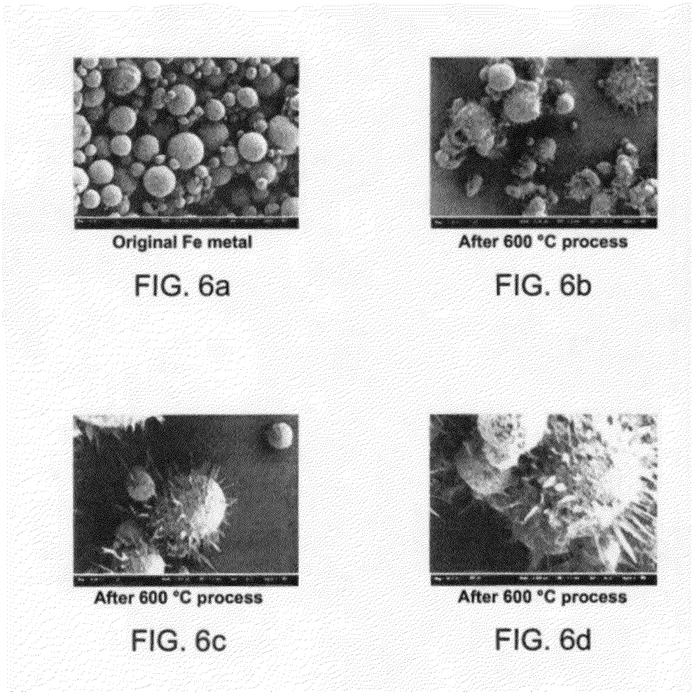

The present invention is applicable to metals other than copper. For example, experiments were done with iron particles. FIG. 6 shows original iron metal particles and then the particles after thermal oxidation at 600° C. showing the growth of nanowires of iron oxide. It is expected that other metals that have multiple oxidation states along with a lattice mismatch with its oxide will result in nanowire growth in an optimal size range of 750 nm-5 μm in starting metal thickness or diameter and thermally oxidized in an optimal temperature range of 300-800° C. Other suitable metals having multiple oxidation states include zinc, beryllium, aluminum, titanium, zirconium, tin, nickel, vanadium, chromium, manganese, cobalt, niobium, molybdenum, ruthenium, rhodium, lead, rhenium, osmium, iridium, platinum, mercury, thallium, bismuth, cerium, praseodymium, samarium, europium, terbium, protactinium, uranium, neptunium, plutonium, americium, berkelium, thulium, ytterbium.

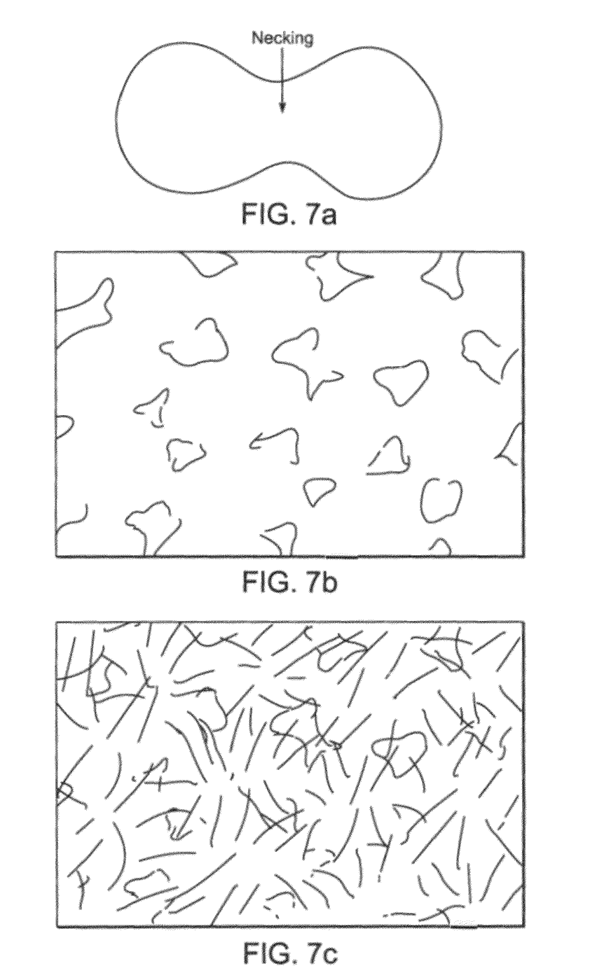

FIGS. 7a, b, and c show an oxidized network of partially sintered metal particles. The particles are first sintered in vacuum, or in an inert atmosphere (or relatively nonreactive gas), to initiate necking, allowing for metal-to-metal contact. Then the particles are sintered in air to impart the secondary nanowire structure on the surface while mostly retaining the high thermal conductivity.

### Experimental Methods

### Thermal Oxidation

The samples were prepared by depositing spherical copper powder (99-99.9% metals basis, Alfa Aesar) with an average particle size of 1 μm, 10 μm, and 50 μm on a flat silicon substrate followed by heating for 30 minutes in a box furnace (Thermolyne Benchtop Muffle Furnace, Thermo Fisher Scientific) that was preset to the desired temperature and operated under ambient air in a fume hood. The 1 μm and 10 μm powder were used as received, and the 50 μm powder was sieved to a size range between 45 μm and 53 μm. The particle size distribution on the 1 μm and 10 μm powders is as follows: for the 1 μm powder, 10% of the particles are at or below 0.47 μm, 50% are at or below 0.75 μm, and 90% are at or below 1.88 μm; for the 10 μm powder, 10% of the particles are at or below 7.34 μm, 50% are at or below 10.17 μm, and 90% are at or below 14.83 μm.

### Thermogravimetric Analysis

Thermogravimetric analysis was performed on a TGA Q50 (TA Instruments) at 600° C. for 1 hr in air. The copper particles were heated to 600° C. in nitrogen, and the gas was switched to air to start the oxidation.

### In-Situ X-Ray Diffraction

A PANalytical X'Pert Pro was used for all experiments. The copper particles were heated to 600° C. in nitrogen, and the gas was switched to an atmosphere consisting of 5% oxygen and 95% nitrogen in order to slow down the rate of the reaction and capture the evolution of the initial process. Every two minutes, the copper sample was scanned between 33-45° with each scan taking two minutes to complete. The raw data was analyzed using the High Score Plus software package.

### FIB Milling And SEM Imaging

A Zeiss NVision 40 was used to mill and image in-situ the resulting structure. An ion current of 1.5 nA at 30 kV was used for milling and a finer mill current of 40 pA at 30 kV for polishing. The SEM images were taken at 2 kV.

It is contemplated that the structures described by the invention disclosed herein will have many applications. For example, the hierarchical micro/nanostructures can be used as a thermal capacitor for thermal storage/regulation in homes or with respect to military attire. The materials of the invention can be filled with a dielectric material to create an ultra capacitor. Further, the particles of the invention can be filled with a material to create a tunable thermal battery.

The hierarchical nanostructures of the invention will also have use in electronics cooling as a new thermal interface material (TIM) especially for spray-impingement of droplets in a heat pipe or a structure filled with a high thermal conductivity material such as a liquid metal. When the application is a thermal interface material, structures of the invention are preferably fabricated in two steps to improve the overall thermal conductivity of the resulting material. First, the particles are sintered in vacuum to initiate necking (to enhance thermal conductivity and increase structural strength). Then the particles are briefly sintered in air to allow the secondary nanowire structure on the surface to form while mostly retaining the thermal conductivity of copper as the primary material comprising the thermal interface material.

The hierarchical nanostructures of the invention may provide a more efficient boiling surface by tuning the size of the surface structures to optimize for high capillarity forces, bubble nucleation, superhydrophillicity, and escape of vapor. In this case, particles are applied to the surface of boiler tubes by thermal spray, dip coating, or other scalable coating processes. If the structures of the invention are applied to zirconium, the boiling surfaces will be particularly useful in nuclear power plants.

The structures of the invention can also form a high-porosity catalyst for use in chemical looping combustion (CLC). The structures of the invention, because of their high surface area, are useful for catalysis and to prevent particle agglomeration with secondary nanowire structure.

Other applications of the invention include use in a lithium ion battery and as a gas sensor. The structures of the invention may be tailored to have desired thermoelectric properties by controlling the oxide thickness of the resulting material by controlling the duration of the oxidation process. When the material is iron, its unique magnetic properties will be useful as MRI contrast agents. The structures of the invention can also form a porous wicking structure for a geothermal heat pipe.

The superscript numbers refer to the references included herewith and the contents of all of these references are incorporated herein by reference.

Supplemental information concerning the process disclosed herein is included in the provisional applications noted earlier to which this non-provisional application claims priority. These provisional applications are incorporated herein by reference.

### REFERENCES

1. M. C. McAlpine, H. Ahmad, D. Wang and J. R. Heath, Nature Materials, 2007, 6, 379-384.
2. P. Chen, G. Shen, H. Chen, Y. Ha, C. Wu, S. Sukcharoenchoke, Y. Fu, J. Liu, A. Facchetti, T. J. Marks, M. E. Thompson and C. Zhou, ACS Nano, 2009, 3, 3383-3390.
3. Z. L. Wang, Science, 2006, 312, 242-246.
4. R. Yan, D. Gargas and P. Yang, Nature Photonics, 2009, 3, 569-576.
5. P. Yang, R. Yan and M. Fardy, Nano letters, 2010, 10, 1529-4536.
6. Y. Xia, P. Yang, Y. Sun, Y. Wu, B. Mayers, B. Gates, Y. Yin, F. Kim and H. Yan, Advanced Materials, 2003, 15, 353-389.
7. A. Dong, R. Tang and W. E. Buhro, J. Am. Chem. Soc, 2007, 129, 12254-12262.
8. T. Mårtensson, C. P. T. Svensson, B. A. Wacaser, M. W. Larsson, W. Seifert, K. Deppert, A. Gustafsson, L. R. Wallenberg and L. Samuelson, Nano Letters, 2004, 4, 1987-1990.
9. T. J. Trentler, K. M. Hickman, S. C. Goel, A. M. Viano, C. G. Patrick and W. Buhro, Science, 1995, 270, 1791-1794.
10. H. Zhou and S. S. Wong, ACS Nano, 2008, 2, 944-958.
11. F. Marabelli, G. B. Parravicini and F. Salghetti-Drioli, Phys. Rev. B, 1995, 52, 1433-1436.
12. F.-M. Chang, S.-L. Cheng, S.-J. Hong, Y.-J. Sheng and H.-K. Tsao, Applied Physics Letters, 2010, 96, 114101.
13. F. Mumm, A. T. J. van Helvoort and P. Sikorski, ACS Nano, 2009, 3, 2647-2652.
14. R. Siriwardane, H. Tian, G. Richards, T. Simonyi and J. Poston, Energy & Fuels, 2009, 23, 3885-3892.
15. Y. Feng, P. M. Rao, D. R. Kim and X. Zheng, Proceedings of the Combustion Institute, 2011, 33, 3169-3175.
16. Y. Feng and X. Zheng, Nano Letters, 2010, 10, 4762-4766.
17. P. M. Rao and X. Zheng, Nano Letters, 2009, 9, 3001-3006
18. C. H. Xu, C. H. Woo and S. Q. Shi, Chemical Physics Letters, 2004, 399, 62-66.
19. X. Jiang, T. Herricks and Y. Xia, Nano Letters, 2002, 2, 1333-1338.
20. J. T. Chen, F. Zhang, J. Wang, G. A. Zhang, B. B. Miao, X. Y. Fan, D. Yan and P. X. Yan, Journal of Alloys and Compounds, 24 Apr. 2008, 454, 268-273.
21. G. P. Peterson, An introduction to heat pipes: modeling, testing, and applications, John Wiley & Sons, Inc., 1994.
22. P. deBock, K. K. Varanasi, P. Chamarthy, T. Deng, A. Kulkarni, B. Rush, B. Russ and S. Weaver, Experimental Investigation of Micro-Nano Heat Pipe Wick Structures, Boston, 2008.
23. M. Kaviany, Principles of Heat Transfer in Porous Media, Springer, 1995.
24. J. A. Weibel, S. V. Garimella and M. T. North, International Journal of Heat and Mass Transfer, 2010, 53, 4204-4215.
25. M. A. Hanlon and H. B. Ma, Journal of Heat Transfer, 2003, 125, 644-652.
26. S. G. Liter and M. Kaviany, International Journal of Heat and Mass Transfer, 2001, 44, 4287-4311.
27. R. Chen, M.-C. Lu, V. Srinivasan, Z. Wang, H. H. Cho and A. Majumdar, Nano Letters, 2009, 9, 548-553.
28. L. Yuan, Y. Wang, R. Mema and G. Zhou, Acta Materialia, 2011, 59, 2491-2500.
29. Y.-W. Park, N.-J. Seong, H.-J. Jung, A. Chanda and S.-G. Yoon, Journal of The Electrochemical Society, 2010, 157, K119.
30. A. M. B. Goncalves, L. C. Campos, A. S. Ferlauto and R. G. Lacerda, Journal of Applied Physics, 2009, 106, 034303.
31. L. S. Huang, S. G. Yang, T. Li, B. X. Gu, Y. W. Du, Y. N. Lu and S. Z. Shi, Journal of Crystal Growth, 2004, 260, 130-135.
32. R. W. Balluffi, S. M. Allen and W. C. Carter, in Kinetics of Materials, John Wiley & Sons, Inc., New Jersey, 2005, ch. 9.
33. F. Aldinger, Acta Metallurgica, 1974, 22, 923-928.
34. Y. Yin, Science, 2004, 304, 711-714.
35. Y. Chang, M. L. Lye and H. C. Zeng, Langmuir, 2005, 21, 3746-3748.
36. H. Kwon, C. J. Love, J. D. Smith, D. Brommer, K. K. Varanasi. 2010. U.S. patent application Ser. No. 12/872353.
37. S. H. Im, U. Jeong and Y. Xia, Nature Materials, 2005, 4, 671-675f.

What is claimed is:

1. Method for making hierarchical structures comprising:
preparing a mixture of relatively smaller metal particles having a size below a threshold for nanowire formation and of relatively larger metal particles having a size above the threshold for nanowire formation; and
oxidizing the mixture at a selected temperature and for a selected time whereby the relatively smaller particles sinter and nanowires grow on the relatively larger particles thereby creating tunable hierarchical structures.

2. The method of claim 1 wherein the particles are copper.

3. The method of claim 1 wherein the particles are iron.

4. The method of claim 2 wherein the threshold is in the range of approximately 2-4 μm.

5. The method of claim 2 wherein the threshold is around 3 μm.

6. The method of claim 2 wherein the selected temperature is within the range of 400-700° C.

7. The method of claim 2 wherein the selected temperature is approximately 600° C.

8. The method of claim 2 wherein the selected time is the range of approximately 30 minutes to 60 minutes.

9. Method for controlling extent of nanowire growth on a metal particle comprising:
selecting a starting particle size with respect to a size threshold for nanowire growth; and
oxidizing the particle at a selected temperature and for a selected time whereby a desired extent of nanowire growth is achieved.

10. Method for making a hollow structure comprising thermally oxidizing a metal particle having a selected diameter.

11. An article comprising nanowires extending from the surface of a particle, the particle including elements having multiple oxidation states.

12. Hollow metal particle made by oxidation, the metal particle characterized by a diffusivity of the metal through its oxide being greater than the diffusivity of oxygen through the oxide.

13. Particle of claim 12 wherein the hollow metal particle further includes nanowires extending from the surface of the particle.

14. The particle of claim 13 used as a coating.

15. The coating of claim 13 wherein the coating includes particles of different sizes forming a porous medium.

16. Method for making a material or coating comprising simultaneous sintering/oxidation of an unoxidized particle in ambient air.

17. Method for forming an oxidized network of partially sintered metal particles comprising:
sintering particles in vacuum, in an inert atmosphere or in a relatively nonreactive gas to initiate necking; and
sintering the particles in air to form nanowires extending from the surface of the particle while retaining high thermal conductivity.

18. Thermal capacitor for thermal storage/regulation comprising a material made by the method of claim 1.

19. Ultracapacitor comprising material made by the method of claim 1 that is filled with a dielectric material.

20. Thermal battery comprising structures made by the method of claim 1 filled with a selected material to create a tunable thermal battery.

21. Thermal interface material comprising the structure made by the method of claim 1 adapted for spray impingement of droplets in a heat pipe.

22. Thermal interface material comprising the structure made by method of claim 1 filled with a high thermal conductivity material.

23. Thermal interface material of claim 22 wherein the high thermal conductivity material is liquid metal.

24. Boiling surface comprising structures made according to claim 1, the size of the surface structure optimized for high capillarity forces, bubble nucleation, superhydrophilicity and escape of vapor.

25. The structure made by the method of claim 1 used for spray cooling.

26. High-porosity hydrocarbon catalyst comprising structure made by the method of claim 1.

27. Gas sensor comprising structure made by the method of claim 1.

28. Contrast agent for MRI comprising structure made by the method of claim 3.

29. Heat pipe comprising structure made by the method of claim 1.

30. Geothermal heat pipe comprising porous wicking structure made by the method of claim 1.

31. Structure made by the method of claim 1 used for carbon sequestration.

32. Battery anode material made by the method of claim 1.

33. Particle comprising a metal oxide outer shell with metal oxide wires extending from the outer shell.

34. The particle of claim 33 having a diameter above a critical size.

35. The particle of claim 33 wherein the particle is hollow.

36. Multiscale structure comprising particles above and below a critical size wherein the particles above the critical size have wires extending from the particle surface.

37. Multiscale structure of claim 36 wherein the particles both above and below the critical size are in intimate metal-to-metal contact.

38. Multiscale structure of claim 37 wherein interstitial spaces are filled with a selected material.

39. The particle of claim 33 or 36 made from a material selected from the group consisting of copper, iron, zinc, beryllium, aluminum, titanium, zirconium, tin, nickel, vanadium, chromium, manganese, cobalt, niobium, molybdenum, ruthenium, rhodium, lead, rhenium, osmium, iridium, platinum, mercury, thallium, bismuth, cerium, praseodymium, samarium, europium, terbium, protactinium, uranium, neptunium, plutonium, americium, berkelium, thulium, ytterbium.
